(19) United States
(12) Patent Application Publication
(10) Pub. No.: US 2008/0255453 A1
(43) Pub. Date: Oct. 16, 2008

(54) ULTRASONIC DIAGNOSTIC APPARATUS

(76) Inventors: Atsuko Matsunaga, Tokyo (JP); Shoko Arita, Tokyo (JP); Koji Miyama, Tokyo (JP)

Correspondence Address: Patrick W. Rasche;Armstrong Teasdale LLP, Suite 2600, One Metropolitan Square, St. Louis, MO, 63102, US

(21) Appl. No.: 12/100,965

(22) Filed: Apr. 10, 2008

(30) Foreign Application Priority Data
Apr. 13, 2007 (JP) 2007-105931

(51) Int. Cl. A61B 8/14

(52) U.S. Cl. 600/445; 600/458

(57) ABSTRACT

An ultrasonic diagnostic apparatus includes: a first ultrasonic probe; a first transmitting/receiving device which drives the first ultrasonic probe to perform scan with an ultrasonic beam; an ultrasonic image generating device which generates an ultrasonic image, based on a signal obtained by the first transmitting/receiving device; an image displaying device which displays the ultrasonic image thereon; a contrast timer which starts clocking in response to a start signal; a second ultrasonic probe; a second transmitting/receiving device which drives the second ultrasonic probe; and a determining device which determines based on a signal obtained by the second transmitting/receiving device whether a contrast agent has flowed in or not, and transmits the start signal to the contrast timer when the contrast agent has flowed in.

### CROSS REFERENCE TO RELATED APPLICATIONS

This application claims the benefit of Japanese Patent Application No. 2007-105931 filed Apr. 13, 2007, and incorporated herein in its entirety.

### BACKGROUND OF THE INVENTION

The subject matter disclosed herein relates to an ultrasonic diagnostic apparatus and an ultrasonic diagnostic apparatus system and, more specifically, to an ultrasonic diagnostic apparatus and an ultrasonic diagnostic apparatus system both capable of starting a contrast timer with accurate timing without placing any load on an operator.

As an ultrasonic diagnostic apparatus, there has been known one having a stop watch function for starting clocking under a start operation of an operator, stopping clocking in accordance with freeze instructions of the operator and displaying elapsed time from its start on the screen. This function is called “contrast timer”.

When the start operation is performed using the stop watch function when a contrast agent is administered into a subject, and the operation for the freeze instructions is carried out at the appropriate time, it is possible to confirm the time subsequent to the administration of the contrast agent elapsed up to the time when a frozen ultrasonic image has been imaged or photographed (refer to, for example, Japanese Unexamined Patent Publication No. 2004-148015).

When, for example, renal contrast imaging is performed in the conventional ultrasonic diagnostic apparatus, the operator holds an ultrasonic probe while applying it to the abdomen of a subject and starts to inject a contrast agent into the antebrachial veins thereof, followed by having done a start operation of the contrast timer when the appropriate opportunity comes.

However, a problem arose in that both the operation of applying the ultrasonic probe to the subject and the operation of performing the start operation of the contrast timer on the ultrasonic diagnostic apparatus would be a burden on the operator. A problem also arose in that the timing provided to start the contrast timer was not kept constant.

### SUMMARY OF THE INVENTION

It is desirable that the problems described previously are solved.

In a first aspect, the invention provides an ultrasonic diagnostic apparatus comprising a first ultrasonic probe, a first transmitting/receiving device which drives the first ultrasonic probe to perform scan with an ultrasonic beam, an ultrasonic image generating device which generates an ultrasonic image, based on a signal obtained by the first transmitting/receiving device, an image displaying device which displays the ultrasonic image thereon, a contrast timer which starts clocking in response to a start signal, a second ultrasonic probe, a second transmitting/receiving device which drives the second ultrasonic probe, and a determining device which determines based on a signal obtained by the second transmitting/receiving device whether a contrast agent has flowed in or not, and transmits the start signal to the contrast timer when the contrast agent has flowed in.

In the ultrasonic diagnostic apparatus according to the first aspect, when, for example, renal contrast imaging is done, an operator holds the first ultrasonic probe while applying it to the abdomen of a subject and fixes the second ultrasonic probe to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a second aspect, the invention provides an ultrasonic diagnostic apparatus wherein in the ultrasonic diagnostic apparatus according to the first aspect, the determining device determines based on a Doppler component of the signal obtained by the second transmitting/receiving device whether the contrast agent has flowed in.

In the ultrasonic diagnostic apparatus according to the second aspect, the inflow of the contrast agent can be detected while capturing a change in Doppler component due to the contrast agent.

In a third aspect, the invention provides an ultrasonic diagnostic apparatus comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a blood flow measuring device, and a determining device which determines based on a signal obtained by the blood flow measuring device whether a contrast agent has flowed in or not, and transmits the start signal to the contrast timer when the contrast agent has flowed in.

In the ultrasonic diagnostic apparatus according to the third aspect, when, for example, renal contrast imaging is performed, an operator holds the ultrasonic probe while applying it to the abdomen of a subject and fixes a sensor for the blood flow measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a fourth aspect, the invention provides an ultrasonic diagnostic apparatus wherein in the ultrasonic diagnostic apparatus according to the third aspect, the blood flow measuring device is a device which measures a blood flow optically.

In the ultrasonic diagnostic apparatus according to the fourth aspect, the inflow of a contrast agent can be detected while capturing a change in light reflected from the blood flow or light transmitted therethrough due to the contrast agent, for example.

In a fifth aspect, the invention provides an ultrasonic diagnostic apparatus wherein in the ultrasonic diagnostic apparatus according to the fourth aspect, the blood flow measuring device is a device which measures a blood flow by laser.

In the ultrasonic diagnostic apparatus according to the fifth aspect, the inflow of a contrast agent can be detected while capturing a change in laser Doppler component due to the contract agent, for example.

In a sixth aspect, the invention provides an ultrasonic diagnostic apparatus comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a blood pressure measuring device, and a determining device which determines based on a signal obtained by the blood pressure measuring device whether a contrast agent has flowed in or not, and transmits the start signal to the contrast timer when the contrast agent has flowed in.

In the ultrasonic diagnostic apparatus according to the sixth aspect, when, for example, renal contrast imaging is performed, an operator holds the ultrasonic probe while applying it to the abdomen of a subject and fixes a sensor for the blood pressure measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a seventh aspect, the invention provides an ultrasonic diagnostic apparatus wherein in the ultrasonic diagnostic apparatus according to the sixth aspect, the blood pressure measuring device is a device which measures blood pressure by mechanical pressure.

In the ultrasonic diagnostic apparatus according to the seventh aspect, the inflow of a contrast agent can be detected while capturing a change in blood pressure due to the contrast agent, for example.

In an eighth aspect, the invention provides an ultrasonic diagnostic apparatus comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a pulse wave measuring device, and a determining device which determines based on a signal obtained by the pulse wave measuring device whether a contrast agent has flowed in or not, and transmits the start signal to the contrast timer when the contrast agent has flowed in.

In the ultrasonic diagnostic apparatus according to the eighth aspect, when, for example, renal contrast imaging is performed, an operator holds the ultrasonic probe while applying it to the abdomen of a subject and fixes a sensor for the pulse wave measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a ninth aspect, the invention provides an ultrasonic diagnostic apparatus wherein in the ultrasonic diagnostic apparatus according to the eighth aspect, the pulse wave measuring device is a device which measures a pulse wave optically.

In the ultrasonic diagnostic apparatus according to the ninth aspect, the inflow of a contrast agent can be detected while capturing a change in light reflected from a blood flow or light transmitted therethrough due to the contrast agent, for example.

In a tenth aspect, the invention provides an ultrasonic diagnostic apparatus system comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a blood flow measuring device, and a determining device which determines based on a signal obtained by the blood flow measuring device whether a contrast agent has flowed in or not, and transmits the start signal to the contrast timer when the contrast agent has flowed in.

In the above configuration, the blood flow measuring device is a device independent of an ultrasonic diagnostic apparatus. Various ones are commercially available.

In the ultrasonic diagnostic apparatus system according to the tenth aspect, when, for example, renal contrast imaging is performed, an operator holds the ultrasonic probe while applying it to the abdomen of a subject and fixes a sensor for the blood flow measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In an eleventh aspect, the invention provides an ultrasonic diagnostic apparatus system wherein in the ultrasonic diagnostic apparatus system according to the tenth aspect, the blood flow measuring device is a device which measures a blood flow by ultrasound.

In the ultrasonic diagnostic apparatus system according to the eleventh aspect, the inflow of a contrast agent can be detected while capturing a change in Doppler component due to the contrast agent.

In a twelfth aspect, the invention provides an ultrasonic diagnostic apparatus system wherein in the ultrasonic diagnostic apparatus system according to the tenth aspect, the blood flow measuring device is a device which measures a blood flow optically.

In the ultrasonic diagnostic apparatus system according to the twelfth aspect, the inflow of a contrast agent can be detected while capturing a change in light reflected from a blood flow or light transmitted therethrough due to the contrast agent, for example.

In a thirteenth aspect, the invention provides an ultrasonic diagnostic apparatus system wherein in the ultrasonic diagnostic apparatus system according to the twelfth aspect, the blood flow measuring device is a device which measures a blood flow by laser.

In the ultrasonic diagnostic apparatus system according to the thirteenth aspect, the inflow of a contrast agent can be detected while capturing a change in laser Doppler component due to the contract agent, for example.

In a fourteenth aspect, the invention provides an ultrasonic diagnostic apparatus system comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a blood pressure measuring device, and a determining device which determines based on a signal obtained by the blood pressure measuring device whether a contrast agent has flowed in or not, and transmits the start signal to the contrast timer when the contrast agent has flowed in.

In the above configuration, the blood pressure measuring device is a device independent of an ultrasonic diagnostic apparatus. Various ones are commercially available.

In the ultrasonic diagnostic apparatus system according to the fourteenth aspect, when, for example, a renal contrast imaging is performed, an operator holds the ultrasonic probe while applying it to the abdomen of a subject and fixes a sensor for the blood pressure measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a fifteenth aspect, the invention provides an ultrasonic diagnostic apparatus system wherein in the ultrasonic diagnostic apparatus system according to the fourteenth aspect, the blood pressure measuring device is a device which measures blood pressure by mechanical pressure.

In the ultrasonic diagnostic apparatus system according to the fifteenth aspect, the inflow of a contrast agent can be detected while capturing a change in blood pressure due to the contrast agent, for example.

In a sixteenth aspect, the invention provides an ultrasonic diagnostic apparatus system comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a pulse wave measuring device, and a determining device which determines based on a signal obtained by the pulse wave measuring device whether a contrast agent has flowed in or not, and transmits the start signal to the contrast timer when the contrast agent has flowed in.

In the above configuration, the pulse wave measuring device is a device independent of an ultrasonic diagnostic apparatus. Various ones are commercially available.

In the ultrasonic diagnostic apparatus system according to the sixteenth aspect, when, for example, renal contrast imaging is performed, an operator holds the ultrasonic probe while applying it to the abdomen of a subject and fixes a sensor for the pulse wave measuring device to the upper arm of the subject. When the injection of the contrast agent into the antebrachial veins is started, the contrast timer is automatically started when the contrast agent has flowed into the brachial veins. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a seventeenth aspect, the invention provides an ultrasonic diagnostic apparatus system wherein in the ultrasonic diagnostic apparatus system according to the sixteenth aspect, an ultrasonic diagnostic apparatus is provided in which the pulse wave measuring device is a device which measures a pulse wave optically.

In the ultrasonic diagnostic apparatus system according to the seventeenth aspect, the inflow of a contrast agent can be detected while capturing a change in light reflected from a blood flow or light transmitted therethrough due to the contrast agent, for example.

In an eighteenth aspect, the invention provides an ultrasonic diagnostic apparatus system comprising an ultrasonic probe, a transmitting/receiving device which drives the ultrasonic probe to perform scan with an ultrasonic beam, an ultrasonic image generating device which generates an ultrasonic image, based on a signal obtained by the transmitting/receiving device, an image displaying device which displays the ultrasonic image thereon, a contrast timer which starts clocking in response to a start signal, a fluid flow measuring device, and a determining device which determines based on a signal obtained by the fluid flow measuring device whether a contrast agent has flowed in or not, and transmits the start signal to the contrast timer when the contrast agent has flowed in.

In the above configuration, the fluid flow measuring device is a device independent of an ultrasonic diagnostic apparatus. Various ones are commercially available.

In the ultrasonic diagnostic apparatus system according to the eighteenth aspect, when, for example, renal contrast imaging is performed, an operator holds the ultrasonic probe while applying it to the abdomen of a subject and fixes a sensor for the fluid flow measuring device to a contrast agent injection tube located in the vicinity of a hole or inlet for injection of the contrast agent into the subject. When the injection of the contrast agent into the antebrachial veins is started, the contrast timer is automatically started when the contrast agent has flowed into the contrast agent injection tube. Thus, since it is not necessary to perform the operation of starting the contrast timer on the ultrasonic diagnostic apparatus, the load on the operator can be lightened. The contrast timer can be started with accurate timing.

Incidentally, the operator is preferably capable of adjusting a delay time between the instant when it is determined that the contrast agent has flowed in and the instant when the start signal is sent to the contrast timer.

In a nineteenth aspect, the invention provides an ultrasonic diagnostic apparatus system where in the ultrasonic diagnostic apparatus system according to the eighteenth aspect, the fluid flow measuring device is a device which measures a fluid flow optically.

In the ultrasonic diagnostic apparatus system according to the nineteenth aspect, the inflow of a contrast agent can be detected while capturing a change in light reflected from the contrast agent injection tube or light transmitted therethrough, for example.

According to an ultrasonic diagnostic apparatus and an ultrasonic diagnostic apparatus system of the invention, a contrast timer of the ultrasonic diagnostic apparatus can be started with accurate timing.

An ultrasonic diagnostic apparatus and an ultrasonic diagnostic apparatus system according to the invention can be used to acquire a TIC (Time Intensity Curve) by contrast imaging, for example.

Further objects and advantages of the present invention will be apparent from the following description of the preferred embodiments of the invention as illustrated in the accompanying drawings.

### BRIEF DESCRIPTION OF THE DRAWINGS

FIG. 1 is a configuration explanatory view showing an ultrasonic diagnostic apparatus according to a first embodiment.

FIG. 2 is a configuration explanatory view illustrating an ultrasonic diagnostic apparatus according to a second embodiment.

FIG. 3 is a configuration explanatory view depicting an ultrasonic diagnostic apparatus according to a third embodiment.

FIG. 4 is a configuration explanatory view showing an ultrasonic diagnostic apparatus according to a fourth embodiment.

FIG. 5 is a configuration explanatory view illustrating an ultrasonic diagnostic apparatus system according to a fifth embodiment.

FIG. 6 is configuration explanatory view showing an ultrasonic diagnostic apparatus system according to a sixth embodiment.

FIG. 7 is a configuration explanatory view illustrating an ultrasonic diagnostic apparatus system according to a seventh embodiment.

FIG. 8 is a configuration explanatory view depicting an ultrasonic diagnostic apparatus system according to an eighth embodiment.

### DETAILED DESCRIPTION OF THE INVENTION

The invention will be explained below in further detail by embodiments illustrated in the drawings. Incidentally, the invention is not limited to or by the embodiments.

### First Embodiment

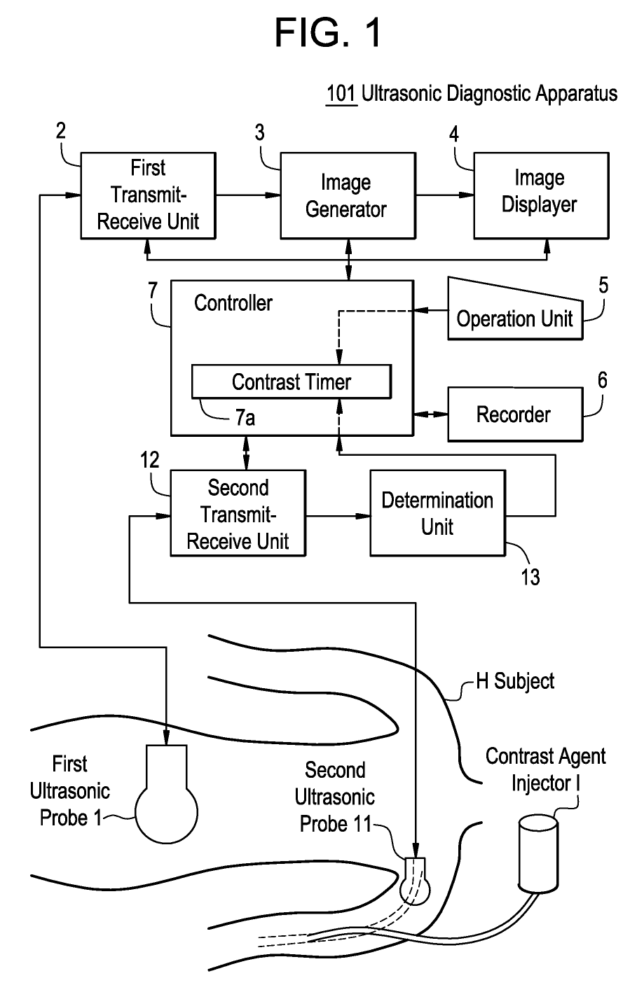

FIG. 1 is a configuration explanatory view showing an ultrasonic diagnostic apparatus 101 according to a first embodiment.

The ultrasonic diagnostic apparatus 101 is equipped with a first ultrasonic probe 1, a first transmit-receive unit 2 which drives the first ultrasonic probe 1 to perform scan within a subject with an ultrasonic beam, an image generator 3 which creates or generates an ultrasonic image, based on a signal obtained from the first transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, the contrast timer 7a included in the controller 7, a second ultrasonic probe 11, a second transmit-receive unit 12 which drives the second ultrasonic probe 11, and a determination unit 13 which determines based on a signal obtained from the second transmit-receive unit 12 whether a contrast agent has flowed in or not, and transmits a start signal to the contract timer 7a when the contrast agent has flowed in.

When, for example, renal contrast imaging is performed, the second ultrasonic probe 11 is fixed onto the forearm of a subject H. The operator holds the first ultrasonic probe 1 while applying it to the abdomen of the subject H and starts to inject a contrast agent into the antebrachial veins of the subject H through a contrast agent injector 1.

The ultrasonic diagnostic apparatus 101 scans the abdomen of the subject H with the first ultrasonic probe 1 to generate an ultrasonic image and displays it. Simultaneously, the ultrasonic diagnostic apparatus 101 detects a Doppler component by the second ultrasonic probe 11.

When the contrast agent flows into the brachial veins, the Doppler component detected by the second ultrasonic probe 11 and outputted from the second transmit-receive unit 12 changes. The determination unit 13 detects it and determines that the contrast agent has flowed in. Then, the operator sends the start signal to the contract timer 7a after the elapse of an adjusted delay time. Thus, the contrast timer 7a is automatically started with accurate timing.

According to the ultrasonic diagnostic apparatus 101 of the first embodiment, the load on the operator can be reduced because the operator needs not to perform the operation of starting the contrast timer 7a on the ultrasonic diagnostic apparatus 101. Since artificial variations inevitable to the start operation by the operator are eliminated, it is possible to always start the contrast timer 7a with accurate timing.

### Second Embodiment

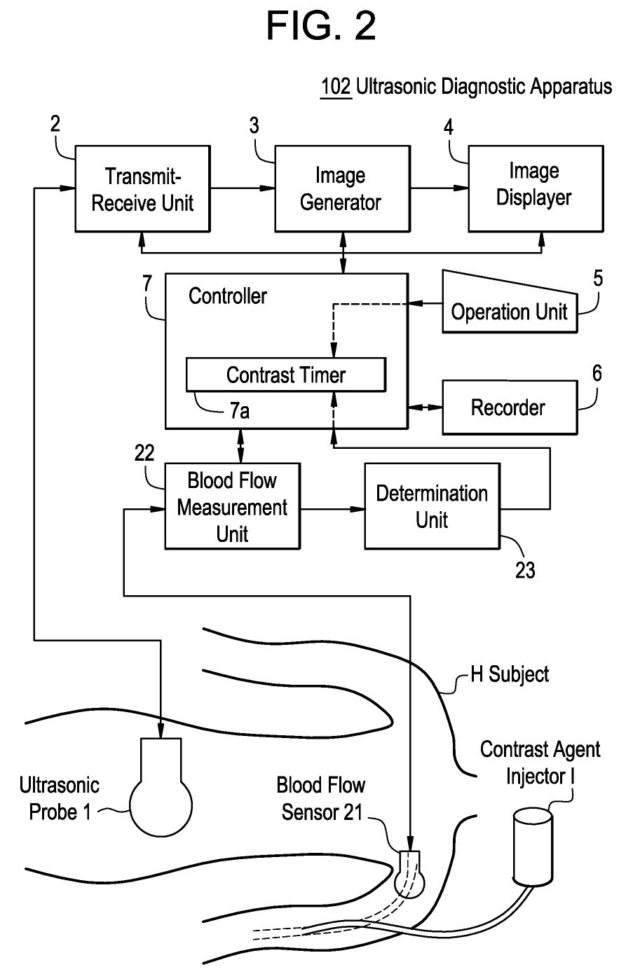

FIG. 2 is a configuration explanatory view showing an ultrasonic diagnostic apparatus 102 according to a second embodiment.

The ultrasonic diagnostic apparatus 102 is equipped with an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, the contrast timer 7a included in the controller 7, a blood flow sensor 21, a blood flow measurement unit 22 which measures a blood flow by the blood flow sensor 21, and a determination unit 23 which determines based on a signal obtained from the blood flow measurement unit 22 whether a contrast agent has flowed in or not, and transmits a start signal to the contract timer 7a when the contrast agent has flowed in.

The blood flow sensor 21 is, for example, an infrared light sensor or a laser Doppler sensor.

When, for example, renal contrast imaging is performed, the blood flow sensor 21 is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 102 scans the abdomen of the subject H with the ultrasonic probe 1 to generate an ultrasonic image and displays it. Simultaneously, the ultrasonic diagnostic apparatus 102 detects a blood flow by the blood flow sensor 21.

When the contrast agent flows into the brachial veins, a blood flow signal detected by the blood flow sensor 21 and outputted from the blood flow measurement unit 22 changes. The determination unit 23 detects it and determines that the contrast agent has flowed in. Then, the operator sends the start signal to the contract timer 7a after the elapse of an adjusted delay time. Thus, the contrast timer 7a is automatically started with accurate timing.

According to the ultrasonic diagnostic apparatus 102 of the second embodiment, the load on the operator can be reduced because the operator needs not to perform the operation of starting the contrast timer 7a on the ultrasonic diagnostic apparatus 102. Since artificial variations inevitable to the start operation by the operator are eliminated, it is possible to always start the contrast timer 7a with accurate timing.

### Third Embodiment

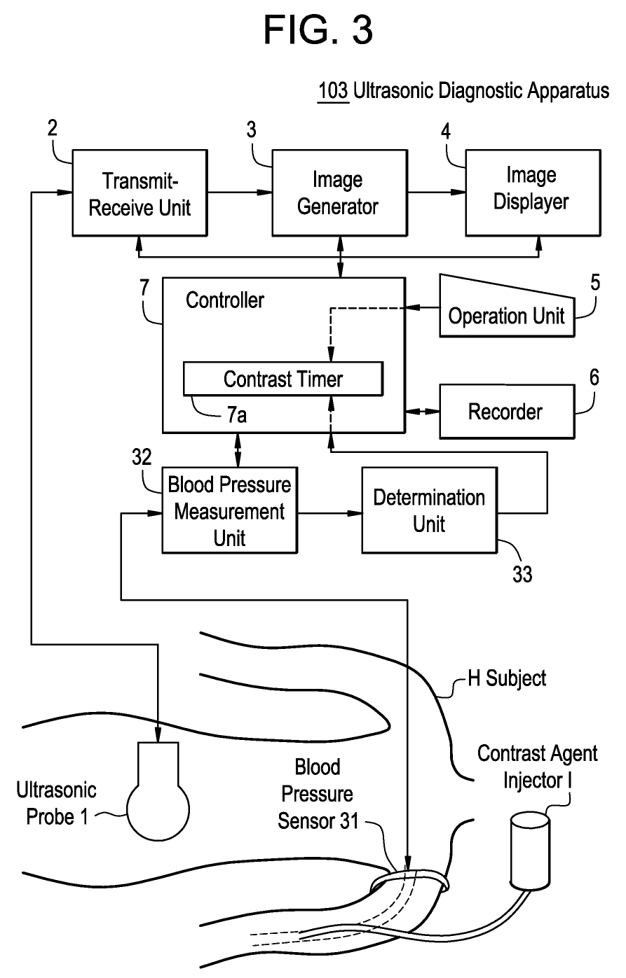

FIG. 3 is a configuration explanatory view showing an ultrasonic diagnostic apparatus 103 according to a third embodiment.

The ultrasonic diagnostic apparatus 103 includes an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, the contrast timer 7a included in the controller 7, a blood pressure sensor 31, a blood pressure measurement unit 32 which measures blood pressure by the blood pressure 31, and a determination unit 33 which determines based on a signal obtained from the blood pressure measurement unit 32 whether a contrast agent has flowed in or not, and transmits a start signal to the contract timer 7a when the contrast agent has flowed in.

The blood pressure sensor 31 is, for example, a pneumatic sensor.

When, for example, renal contrast imaging is performed, the blood pressure sensor 31 is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 103 scans the abdomen of the subject H with the ultrasonic probe 1 to generate an ultrasonic image and displays it. At the same time, the ultrasonic diagnostic apparatus 102 detects blood pressure by the blood pressure sensor 31.

When the contrast agent flows into the brachial veins, a blood pressure signal detected by the blood pressure sensor 31 and outputted from the blood pressure measurement unit 32 changes. The determination unit 33 detects it and determines that the contrast agent has flowed in. Then, the operator sends the start signal to the contract timer 7a after the elapse of an adjusted delay time. Thus, the contrast timer 7a is automatically started with accurate timing.

According to the ultrasonic diagnostic apparatus 103 of the third embodiment, the load on the operator can be lightened because the operator needs not to perform the operation of starting the contrast timer 7a on the ultrasonic diagnostic apparatus 103. Since artificial variations inevitable to the start operation by the operator are eliminated, it is possible to always start the contrast timer 7a with accurate timing.

### Fourth Embodiment

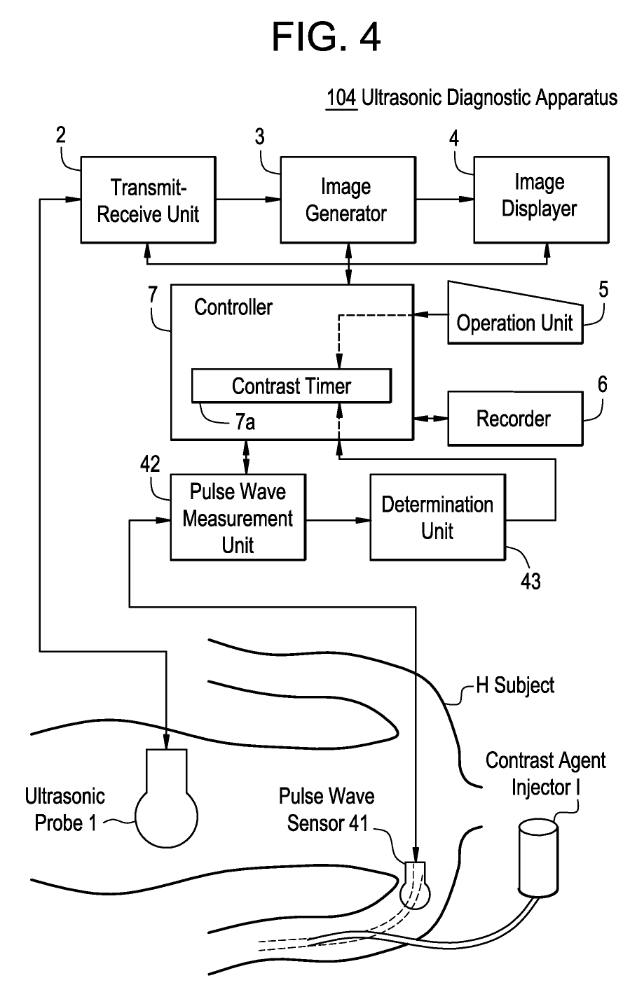

FIG. 4 is a configuration explanatory view showing an ultrasonic diagnostic apparatus 104 according to a fourth embodiment.

The ultrasonic diagnostic apparatus 104 includes an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, the contrast timer 7a included in the controller 7, a pulse wave sensor 41, a pulse wave measurement unit 42 which measures a pulse wave by the pulse wave sensor 41, and a determination unit 43 which determines based on a signal obtained from the pulse wave measurement unit 42 whether a contrast agent has flowed in or not, and transmits a start signal to the contract timer 7a when the contrast agent has flowed in.

The pulse wave sensor 41 is, for example, an infrared light sensor or a laser Doppler sensor.

When, for example, renal contrast imaging is performed, the pulse wave sensor 41 is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 104 scans the abdomen of the subject H with the ultrasonic probe 1 to generate an ultrasonic image and displays it. Simultaneously, the ultrasonic diagnostic apparatus 104 detects a pulse wave by the pulse wave sensor 41.

When the contrast agent flows into the brachial veins, a pulse wave signal detected by the pulse wave sensor 41 and outputted from the pulse wave measurement unit 42 changes. The determination unit 43 detects it and determines that the contrast agent has flowed in. Then, the operator sends the start signal to the contract timer 7a after the elapse of an adjusted delay time. Thus, the contrast timer 7a is automatically started with accurate timing.

According to the ultrasonic diagnostic apparatus 104 of the fourth embodiment, the load on the operator can be reduced because the operator needs not to perform the operation of starting the contrast timer 7a on the ultrasonic diagnostic apparatus 104. Since artificial variations inevitable to the start operation by the operator are eliminated, it is possible to always start the contrast timer 7a with accurate timing.

### Fifth Embodiment

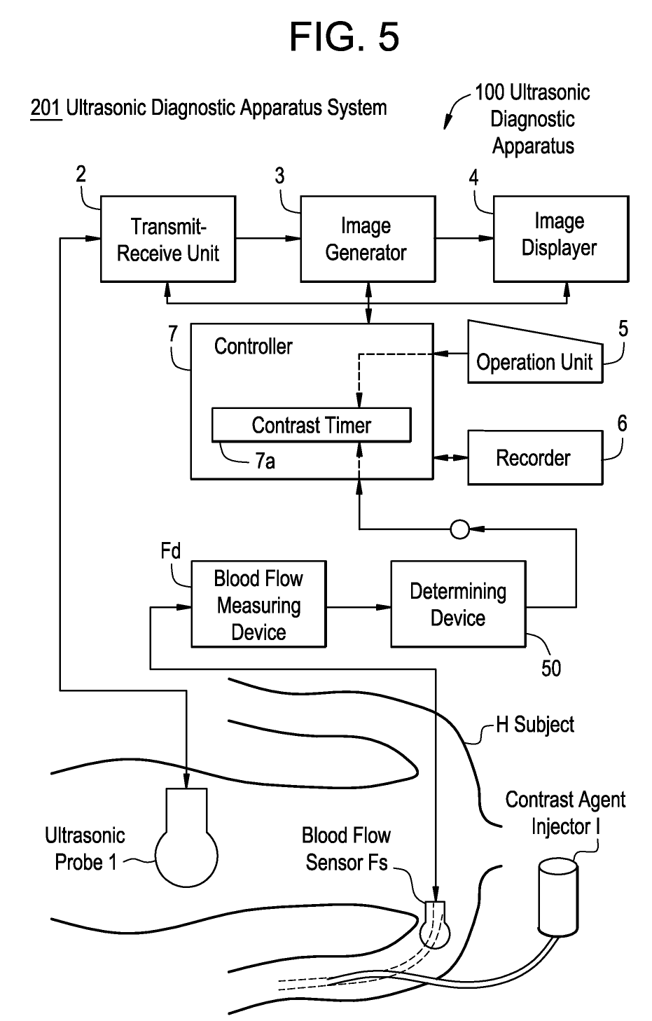

FIG. 5 is a configuration explanatory view showing an ultrasonic diagnostic apparatus system 201 according to a fifth embodiment.

The ultrasonic diagnostic apparatus system 201 includes an ultrasonic diagnostic apparatus 100 equipped with an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, and the contrast timer 7a included in the controller 7; a blood flow measuring device Fd which measures a blood flow by a blood flow sensor Fs; and a determining device 50 which determines based on a signal obtained from the blood flow measuring device Fd whether a contrast agent has flowed in or not, and transmits a start signal for the contract timer 7a to the ultrasonic diagnostic apparatus 100 when the contrast agent has flowed in.

The blood flow measuring device Fd is a device independent of the ultrasonic diagnostic apparatus 100. A commercially available device may be used therefore.

The blood flow sensor Fs is, for example, an infrared light sensor or a laser Doppler sensor.

When, for example, renal contrast imaging is performed, the blood flow sensor Fs is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 100 scans the abdomen of the subject H with the ultrasonic probe 1 to generate an ultrasonic image and displays it.

The blood flow measuring device Fd detects a blood flow by means of the blood flow sensor Fs.

When the contrast agent flows into the brachial veins, a blood flow signal detected by the blood flow sensor Fs and outputted from the blood flow measuring device Fd changes. The determining device 40 detects it and determines that the contrast agent has flowed in. Then, the operator sends the start signal for the contrast timer 7a to the ultrasonic diagnostic apparatus 100 after the elapse of an adjusted delay time. Thus, the contrast timer 7a of the ultrasonic diagnostic apparatus 100 is automatically started with accurate timing.

According to the ultrasonic diagnostic apparatus system 201 of the fifth embodiment, the load on the operator can be lightened because the operator needs not to perform the operation of starting the contrast timer 7a on the ultrasonic diagnostic apparatus 100. Since artificial variations inevitable to the start operation by the operator are eliminated, it is possible to always start the contrast timer 7a with accurate timing.

### Sixth Embodiment

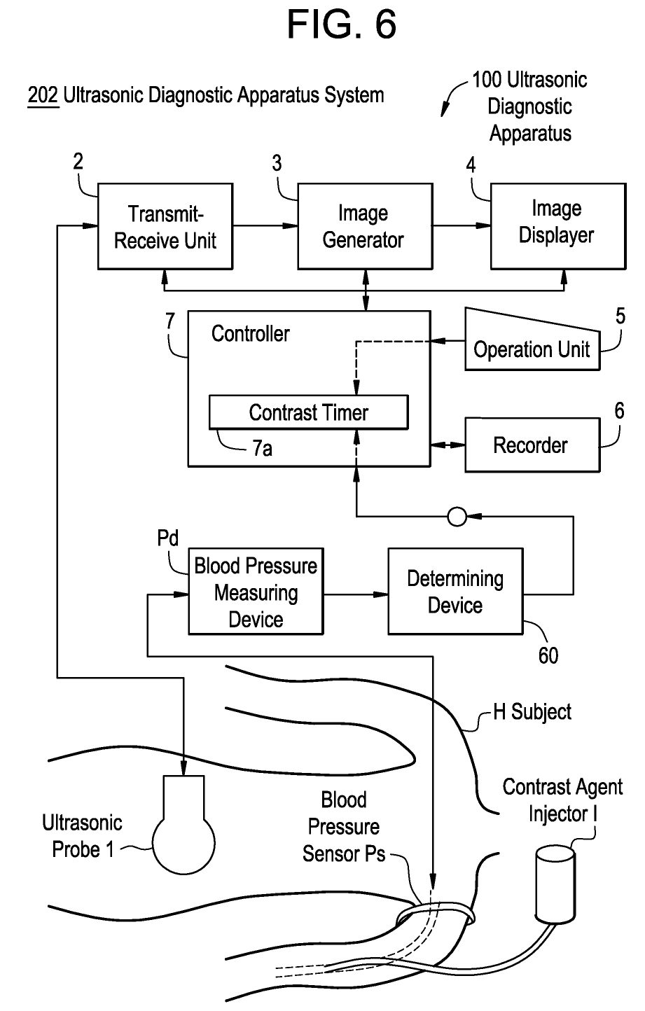

FIG. 6 is a configuration explanatory view showing an ultrasonic diagnostic apparatus system 202 according to a sixth embodiment.

The ultrasonic diagnostic apparatus system 202 includes an ultrasonic diagnostic apparatus 100 equipped with an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, and the contrast timer 7a included in the controller 7; a blood pressure measuring device Pd which measures blood pressure by a blood pressure sensor Ps; and a determining device 60 which determines based on a signal obtained from the blood pressure measuring device Pd whether a contrast agent has flowed in or not, and transmits a start signal for the contract timer 7a to the ultrasonic diagnostic apparatus 100 when the contrast agent has flowed in.

The blood pressure measuring device Pd is a device independent of the ultrasonic diagnostic apparatus 100. A commercially available device may be used therefore.

The blood pressure sensor Ps is, for example, a pneumatic sensor.

When, for example, renal contrast imaging is performed, the blood pressure sensor Ps is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 100 scans the abdomen of the subject H with the ultrasonic probe 1 to generate an ultrasonic image and displays it.

The blood pressure measuring device Pd detects blood pressure by means of the blood pressure sensor Ps.

When the contrast agent flows into the brachial veins, a blood pressure signal detected by the blood pressure sensor Ps and outputted from the blood pressure measuring device Pd changes. The determining device 60 detects it and determines that the contrast agent has flowed in. Then, the operator sends the start signal for the contrast timer 7a to the ultrasonic diagnostic apparatus 100 after the elapse of an adjusted delay time. Thus, the contrast timer 7a of the ultrasonic diagnostic apparatus 100 is automatically started with accurate timing.

According to the ultrasonic diagnostic apparatus system 202 of the sixth embodiment, the load on the operator can be lightened because the operator needs not to perform the operation of starting the contrast timer 7a on the ultrasonic diagnostic apparatus 100. Since artificial variations inevitable to the start operation by the operator are eliminated, it is possible to always start the contrast timer 7a with accurate timing.

### Seventh Embodiment

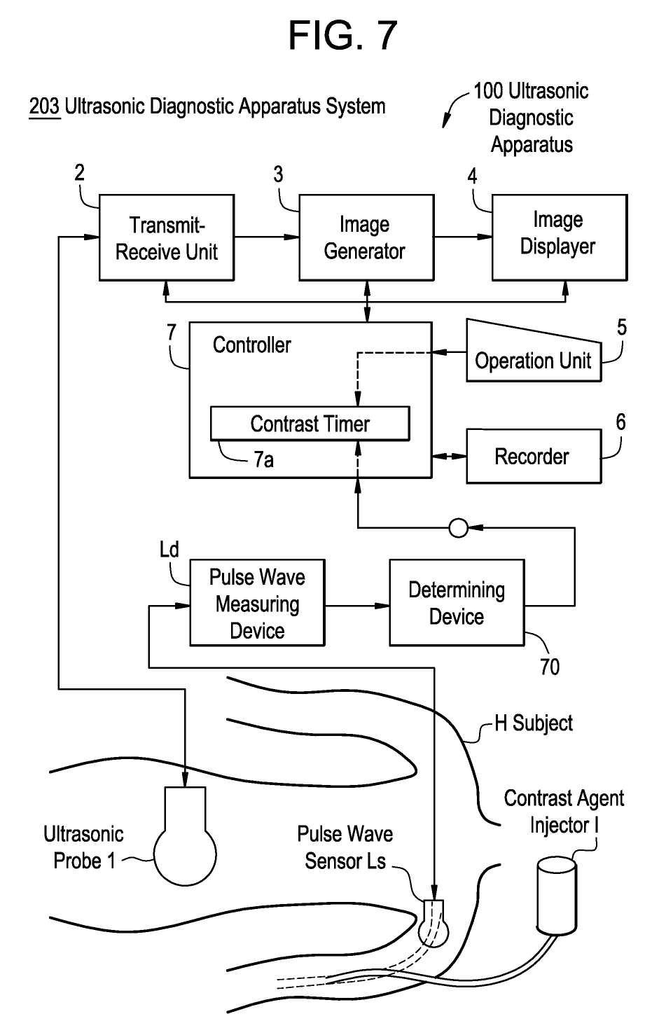

FIG. 7 is a configuration explanatory view showing an ultrasonic diagnostic apparatus system 203 according to a seventh embodiment.

The ultrasonic diagnostic apparatus system 203 includes an ultrasonic diagnostic apparatus 100 equipped with an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, and the contrast timer 7a included in the controller 7; a pulse wave measuring device Ld which measures a pulse wave by a pulse wave sensor Ls; and a determining device 70 which determines based on a signal obtained from the pulse wave measuring device Ld whether a contrast agent has flowed in or not, and transmits a start signal for the contract timer 7a to the ultrasonic diagnostic apparatus 100 when the contrast agent has flowed in.

The pulse wave measuring device Ld is a device independent of the ultrasonic diagnostic apparatus 100. A commercially available device may be used therefore.

The pulse wave sensor Ls is, for example, an infrared light sensor or a laser Doppler sensor.

When, for example, renal contrast imaging is performed, the pulse wave sensor Ls is fixed onto the upper arm of a subject H. The operator holds the ultrasonic probe 1 while applying it to the abdomen of the subject H and starts to inject a contrast agent into the antebrachial veins of the subject H through a contrast agent injector I.

The ultrasonic diagnostic apparatus 100 scans the abdomen of the subject H with the ultrasonic probe 1 to generate an ultrasonic image and displays it.

The pulse wave measuring device Ld detects a pulse wave by means of the pulse wave sensor Ls.

When the contrast agent flows into the brachial veins, a pulse wave signal detected by the pulse wave sensor Ls and outputted from the pulse wave measuring device Ld changes. The determining device 70 detects it and determines that the contrast agent has flowed in. Then, the operator sends the start signal for the contrast timer 7a to the ultrasonic diagnostic apparatus 100 after the elapse of an adjusted delay time. Thus, the contrast timer 7a of the ultrasonic diagnostic apparatus 100 is automatically started with accurate timing.

According to the ultrasonic diagnostic apparatus system 203 of the seventh embodiment, the load on the operator can be lightened because the operator needs not to perform the operation of starting the contrast timer 7a on the ultrasonic diagnostic apparatus 100. Since artificial variations inevitable to the start operation by the operator are avoided, the contrast timer 7a can always be started with accurate timing.

### Eighth Embodiment

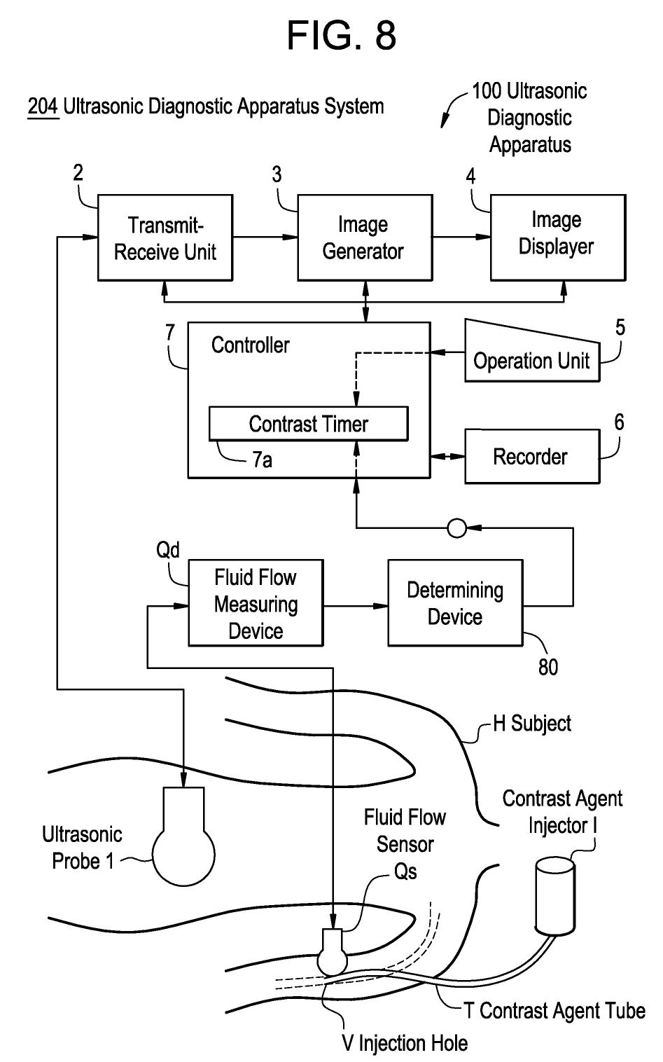

FIG. 8 is a configuration explanatory view showing an ultrasonic diagnostic apparatus system 204 according to an eighth embodiment.

The ultrasonic diagnostic apparatus system 204 includes an ultrasonic diagnostic apparatus 100 equipped with an ultrasonic probe 1, a transmit-receive unit 2 which drives the ultrasonic probe 1 to perform scan within a subject with an ultrasonic beam, an image generator 3 which creates or generates an ultrasonic image, based on a signal obtained from the transmit-receive unit 2, an image displayer 4 which displays the ultrasonic image and the like thereon, an operation unit 5 for allowing an operator to give instructions such as a start operation of a contrast timer 7a and data, a recorder 6 which records the ultrasonic image and the like therein, a controller 7 which controls its entirety, and the contrast timer 7a included in the controller 7; a fluid flow measuring device Qd which measures a fluid flow by a fluid flow sensor Qs; and a determining device 80 which determines based on a signal obtained from the fluid flow measuring device Qd whether a contrast agent has flowed in or not, and transmits a start signal for the contract timer 7a to the ultrasonic diagnostic apparatus 100 when the contrast agent has flowed in.

The fluid flow measuring device Qd is a device independent of the ultrasonic diagnostic apparatus 100. A commercially available device may be used therefore.

The fluid flow sensor Qs is, for example, an infrared light sensor.

When, for example, renal contrast imaging is performed, the fluid flow sensor Qs is fixed to a contrast agent tube T located in the vicinity of a hole or inlet V for injection of the contrast agent into the subject H. The operator holds the ultrasonic probe 1 while applying it to the abdomen of the subject H and starts to inject the contrast agent into the antebrachial veins of the subject H through a contrast agent injector I.

The ultrasonic diagnostic apparatus 100 scans the abdomen of the subject H with the ultrasonic probe 1 to generate an ultrasonic image and displays it.

The fluid flow measuring device Qd detects a fluid flow by means of the fluid flow sensor Qs.

When the contrast agent flows into the contrast agent tube T, a fluid flow signal detected by the fluid flow sensor Qs and outputted from the fluid flow measuring device Qd changes. The determining device 80 detects it and determines that the contrast agent has flowed in. Then, the operator sends the start signal for the contrast timer 7a to the ultrasonic diagnostic apparatus 100 after the elapse of an adjusted delay time. Thus, the contrast timer 7a of the ultrasonic diagnostic apparatus 100 is automatically started with accurate timing.

According to the ultrasonic diagnostic apparatus system 204 of the eighth embodiment, the load on the operator can be lightened because the operator needs not to perform the operation of starting the contrast timer 7a on the ultrasonic diagnostic apparatus 100. Since artificial variations inevitable to the start operation by the operator are avoided, the contrast timer 7a can always be started with accurate timing.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a first ultrasonic probe;
a first transmitting/receiving device configured to drive the first ultrasonic probe during a scan of a subject with an ultrasonic beam;
an ultrasonic image generating device configured to generate an ultrasonic image based on a signal obtained by the first transmitting/receiving device;
an image displaying device configured to display the ultrasonic image generated by the ultrasonic image generating, device;
a contrast timer configured to start a clock in response to a start signal;
a second ultrasonic probe;
a second transmitting/receiving device configured to drive the second ultrasonic probe; and
a determining device configured to determine whether a contrast agent has flowed into the subject based on a signal obtained by the second transmitting/receiving device and to transmit the start signal to the contrast timer when the contrast agent is determined to have flowed into the subject.

2. The ultrasonic diagnostic apparatus according to claim 1, wherein the determining device is configured to determine whether the contrast agent has flowed into the subject based on a Doppler component of the signal obtained by the second transmitting/receiving device.

3. An ultrasonic diagnostic apparatus comprising:
an ultrasonic probe;
a transmitting/receiving device configured to drive the ultrasonic probe during a scan of a subject with an ultrasonic beam;
an ultrasonic image generating device configured to generate an ultrasonic image based on a signal obtained by the transmitting/receiving device;
an image displaying device configured to display the ultrasonic image generated by the ultrasonic image generating device;
a contrast timer configured to start a clock in response to a start signal;
a blood flow measuring device; and
a determining device configured to determine whether a contrast agent has flowed into the subject based on a signal obtained by the blood flow measuring device and to transmit the start signal to the contrast timer when the contrast agent is determined to have flowed into the subject.

4. The ultrasonic diagnostic apparatus according to claim 3, wherein the blood flow measuring device comprises a device configured to measure a blood flow optically.

5. The ultrasonic diagnostic apparatus according to claim 4, wherein the blood flow measuring device comprises a device configured to measure blood flow by laser.

6. An ultrasonic diagnostic apparatus comprising:
an ultrasonic probe;
a transmitting/receiving device configured to drive the ultrasonic probe during a scan of a subject with an ultrasonic beam;
an ultrasonic image generating device configured to generate an ultrasonic image based on a signal obtained by the transmitting/receiving device;
an image displaying device configured to display the ultrasonic image generated by the ultrasonic image generating device;
a contrast timer configured to start a clock in response to a start signal;
a blood pressure measuring device; and
a determining device configured to determine whether a contrast agent has flowed into the subject based on a signal obtained by the blood pressure measuring device and to transmit the start signal to the contrast timer when the contrast agent is determined to have flowed into the subject.

7. The ultrasonic diagnostic apparatus according to claim 6, wherein the blood pressure measuring device comprises a device configured to measure blood pressure by mechanical pressure.

8. An ultrasonic diagnostic apparatus comprising:
an ultrasonic probe;
a transmitting/receiving device configured to drive the ultrasonic probe during a scan of a subject with an ultrasonic beam;
an ultrasonic image generating device configured to generate an ultrasonic image based on a signal obtained by the transmitting/receiving device;
an image displaying device configured to display the ultrasonic image generated by the ultrasonic image generating device;
a contrast timer configured to start a clock in response to a start signal;
a pulse wave measuring device; and
a determining device configured to determine whether a contrast agent has flowed into the subject based on a signal obtained by the pulse wave measuring device and to transmit the start signal to the contrast timer when the contrast agent is determined to have flowed into the subject.

9. The ultrasonic diagnostic apparatus according to claim 8, wherein the pulse wave measuring device comprises a device configured to measure a pulse wave optically.

10. The ultrasonic diagnostic apparatus according to claim 1, wherein the determining device is configured to enable an operator to set a delay time between when the determining device determines that contrast agent has flowed into the subject and when the determining device transmits the start signal to the contrast timer.

11. The ultrasonic diagnostic apparatus according to claim 1, wherein the ultrasonic diagnostic apparatus is configured to inject the contrast agent into the subject's antebrachial vein, and wherein the determining device is configured to transmit the start signal to the contrast timer when the contrast agent is determined to have flowed into the subject's brachial veins.

12. The ultrasonic diagnostic apparatus according to claim 3, wherein the determining device is configured to enable an operator to set a delay time between when the determining device determines that contrast agent has flowed into the subject and when the determining device transmits the start signal to the contrast timer.

13. The ultrasonic diagnostic apparatus according to claim 3, wherein the blood flow measuring device comprises a device configured to measure blood flow by ultrasound.

14. The ultrasonic diagnostic apparatus according to claim 13, wherein the signal transmitted from the blood flow measuring device to the determining device is based on a change in a Doppler component due to inflow of the contrast agent.

15. The ultrasonic diagnostic apparatus according to claim 4, wherein the signal transmitted from the blood flow measuring device to the determining device is based on a change in light reflected from blood flow through the subject due to inflow of the contrast agent.

16. The ultrasonic diagnostic apparatus according to claim 5, wherein the signal transmitted from the blood flow measuring device to the determining device is based on a change in a laser Doppler component due to inflow of the contrast agent.

17. The ultrasonic diagnostic apparatus according to claim 6, wherein the determining device is configured to enable an operator to set a delay time between when the determining device determines that contrast agent has flowed into the subject and when the determining device transmits the start signal to the contrast timer.

18. The ultrasonic diagnostic apparatus according to claim 6, wherein the signal transmitted from the blood pressure measuring device to the determining device is based on a change in a blood pressure due to inflow of the contrast agent.

19. The ultrasonic diagnostic apparatus according to claim 8, wherein the determining device is configured to enable an operator to set a delay time between when the determining device determines that contrast agent has flowed into the subject and when the determining device transmits the start signal to the contrast timer.

20. The ultrasonic diagnostic apparatus according to claim 8, wherein the signal transmitted from the pulse wave measuring device to the determining device is based on a change in light reflected from blood flow through the subject due to inflow of the contrast agent.
